(19) United States
(12) Patent Application Publication
(10) Pub. No.: US 2014/0030822 A1
(43) Pub. Date: Jan. 30, 2014

(54) METHOD FOR IMMOBILIZING AN ANTIBODY ON A SELF-ASSEMBLED MONOLAYER

(71) Applicant: PANASONIC CORPORATION, Osaka (JP)

(72) Inventor: Yukari HATAOKA, Kyoto (JP)

(73) Assignee: PANASONIC CORPORATION, Osaka (JP)

(21) Appl. No.: 14/042,656

(22) Filed: Sep. 30, 2013

Related U.S. Application Data
(63) Continuation of application No. PCT/JP2011/005037, filed on Sep. 7, 2011.

(30) Foreign Application Priority Data
Jun. 10, 2011 (JP) 2011-129893

(51) Int. Cl. G01N 33/553

(52) U.S. Cl. CPC G01N 33/553
USPC 436/501

(57) ABSTRACT

The method of the present disclosure is characterized by that one molecule of the amino acid is interposed between the self-assembled monolayer and the molecule of the antibody. For example, a method for immobilizing an antibody on a self-assembled monolayer is provided and the method including the following steps (a) and (b) in this order: a step (a) of preparing a substrate comprising one molecule of an amino acid and the self-assembled monolayer and a step (b) of supplying the antibody to the substrate to form a peptide bond represented by a predetermined chemical formula as a result of reaction between the carboxyl group of the one molecule of the amino acid and the amino group of the antibody.

### CROSS-REFERENCE TO RELATED APPLICATION

This application is a continuation of International Application No. PCT/JP2011/005037, filed on Sep. 7, 2011, which in turn claims the benefit of Japanese Patent Application No. 2011-129893, filed on Jun. 10, 2011, the disclosures of which Applications are incorporated by reference herein.

### TECHNICAL FIELD

The technologies herein relate to a method for immobilizing an antibody on a self-assembled monolayer.

### BACKGROUND

A biosensor is used to detect or quantify an antigen contained in a sample. A high affinity between an antigen and an antibody can be used in the biosensor. More specifically, an antibody is immobilized on the biosensor. When the antigen is supplied to the biosensor, the antigen is immobilized on the biosensor due to the high affinity between the antigen and the antibody.

International Publication No. WO00/04382 (Patent Literature 1) discloses a conventional biosensor comprising an antibody. Patent Literature 1 corresponds to Japanese Unexamined Patent Publication (Translation of PCT Application) No. 2002-520618 (see, e.g. Page 24 lines 23-26, Page 25 lines 3-20, Page 25 line 27-Page 26 line 13, and Page 26 lines 14-22, Page 28 lines 21-23, Page 32 lines 3-29, Page 35 line 21-Page 36 line 21 of Patent Literature 1 or paragraphs [0080], [0082], [0084], [0085], [0095], [0109], [0118], and [0119] of the corresponding publication). FIG. 2 shows a biosensor disclosed in FIG. 7 of Patent Literature 1.

According to the description regarding FIG. 7 of Patent Literature 1, the biosensor is used for screening an activity of a biomolecule. The biosensor comprises a monolayer 7, an affinity tag 8, an adaptor molecule 9, and a protein 10. The monolayer 7 is composed of a self-assembled monolayer represented by chemical formula: X—R—Y (see Page 24 lines 23-26, Page 25 lines 3-20, Page 25 line 27-Page 26 line 13, and Page 26 lines 14-22 of Patent Literature 1; or paragraphs 0080, 0082, 0084 and 0085 of the corresponding publication). Examples of X, R, and Y are HS—, an alkane, and a carboxyl group, respectively (see Page 25 lines 3-20, Page 25 lines 27-Page 26 line 13, and Page 28 lines 21-23 of Patent Literature 1; or paragraphs 0084, 0085, and 0095 of the corresponding publication).

### SUMMARY

In order to improve the detection sensitivity or the quantification accuracy of the antigen, it is required to increase an amount of the antibody to be immobilized on the biosensor.

The present inventor has discovered that the amount of the antibody immobilized per unit area was increased significantly by binding one molecule amino acid to a self-assembled monolayer and then immobilizing the antibody. The disclosure has been provided on the basis of this discovery.

One non-limiting and exemplary embodiment provides a method for increasing an amount of the antibody to be immobilized on the self-assembled monolayer, and a sensor having the antibody immobilized in accordance with the same method.

Such an embodiment may be indicated, for example, in the following items 1 to 21:

(1) A method for immobilizing an antibody on a self-assembled monolayer, the method comprising steps of:

a step (a) of preparing a substrate comprising one molecule of an amino acid and the self-assembled monolayer, wherein

the one molecule of the amino acid is bound to the self-assembled monolayer through a peptide bond represented by the following chemical formula (I):

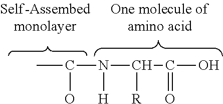

(R represents side chain of one molecule of the amino acid)

the one molecule of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine, and

a step (b) of supplying the antibody to the substrate to form a peptide bond represented by the following chemical formula (II) as a result of reaction between the carboxyl group of the one molecule of the amino acid and the amino group of the antibody.

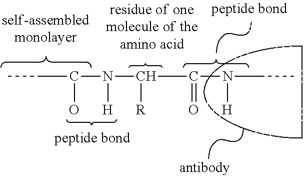

(R represents side chain of one molecule of the amino acid)

(2) The method according to item 1, wherein the step (a) comprises the following steps (a1) and (a2):

a step (a1) of preparing a substrate comprising a self-assembled monolayer on the surface thereof, the self-assembled monolayer having a carboxyl group at one end, and

a step (a2) of supplying the one molecule of the amino acid to form a peptide bond represented by the chemical formula (I) as a result of reaction between the carboxyl group of the one end of the self-assembled monolayer and the amino group of the one molecule of the amino acid.

(3) The method according to item 1, further comprising the following step (ab) between the step (a) and the step (b):

a step (ab) of activating the carboxyl group of the one molecule of the amino acid with a mixture of N-Hydroxysuccinimide and 1-ethyl-3-(3-dimethylaminopropyl)carbodiimide hydrochloride.

(4) The method according to item 2, further comprising the following step (a1a) between the step (a1) and the step (a2):

a step (a1a) of activating the carboxyl group of the self-assembled monolayer with a mixture of N-Hydroxysuccinimide and 1-ethyl-3-(3-dimethylaminopropyl)carbodiimide hydrochloride.

(5) The method according to item 1, wherein the chemical formula (II) is represented by the following chemical formula (III).

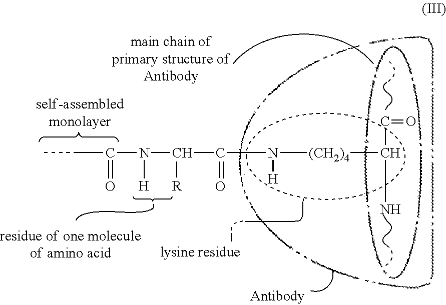

(R represents side chain of one molecule of the amino acid)

(6) The method according to item 1, wherein the one molecule of the amino acid is selected from the group consisting of histidine, cysteine, lysine, phenylalanine, glycine, tryptophan, methionine, serine, asparagine, tyrosine, alanine, glutamic acid, threonine, leucine, valine, and isoleucine.

(7) The method according to item 1, wherein the one molecule of the amino acid is selected from the group consisting of histidine, cysteine, lysine, phenylalanine, glycine, tryptophan, methionine, serine, asparagine, tyrosine, alanine, glutamic acid, and threonine.

(8) The method according to item 1, wherein the one molecule of the amino acid is selected from the group consisting of histidine, cysteine, lysine, phenylalanine, glycine, tryptophan, methionine, serine, asparagine, tyrosine, alanine, and glutamic acid.

(9) The method according to item 1, wherein the one molecule of the amino acid is selected from the group consisting of histidine, cysteine, lysine, phenylalanine, glycine, tryptophan, methionine, serine, asparagine, and tyrosine.

(10) A sensor comprising a self-assembled monolayer, one molecule of an amino acid, and an antibody, wherein

the one molecule of the amino acid is interposed between the self-assembled monolayer and the antibody,

the antibody is bound to the self-assembled monolayer through two peptide bonds represented by the following chemical formula (II),

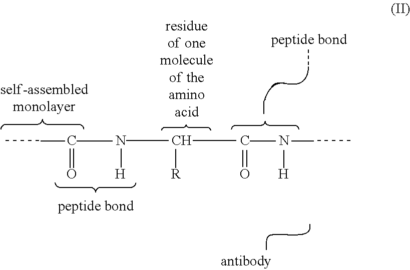

(R represents side chain of one molecule of the amino acid)

the one molecule of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine.

(11) The sensor according to item 10, wherein the chemical formula (II) is represented by the following chemical formula (III).

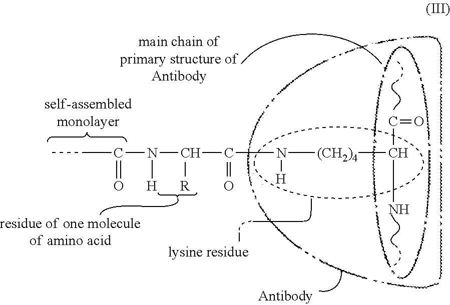

(R represents side chain of one molecule of the amino acid)

(12) The sensor according to item 10, wherein the one molecule of the amino acid is selected from the group consisting of histidine, cysteine, lysine, phenylalanine, glycine, tryptophan, methionine, serine, asparagine, tyrosine, alanine, glutamic acid, threonine, leucine, valine, and isoleucine.

(13) The sensor according to item 10, wherein the one molecule of the amino acid is selected from the group consisting of histidine, cysteine, lysine, phenylalanine, glycine, tryptophan, methionine, serine, asparagine, tyrosine, alanine, glutamic acid, and threonine.

(14) The sensor according to item 10, wherein the one molecule of the amino acid is selected from the group consisting of histidine, cysteine, lysine, phenylalanine, glycine, tryptophan, methionine, serine, asparagine, tyrosine, alanine, and glutamic acid.

(15) The sensor according to item 10, wherein the one molecule of the amino acid is selected from the group consisting of histidine, cysteine, lysine, phenylalanine, glycine, tryptophan, methionine, serine, asparagine, and tyrosine.

(16) A method for detecting or quantifying an antigen contained in a sample with use of a sensor, the method comprising steps of:

a step (a) of preparing the sensor comprising a self-assembled monolayer, one molecule of an amino acid, and an antibody, wherein

the one molecule of the amino acid is interposed between the self-assembled monolayer and the antibody,

the antibody is bound to the self-assembled monolayer through two peptide bonds represented by the following chemical formula (II),

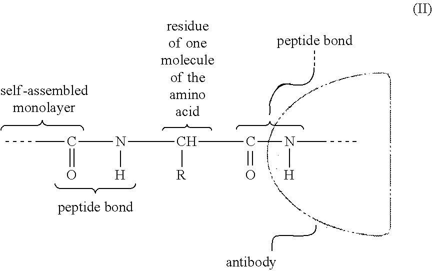

(R represents side chain of one molecule of the amino acid)

the one molecule of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine,

a step (b) of supplying the sample to the sensor to bind the antigen to the antibody, and

a step (c) of detecting the antigen bound in the step (b) or quantify the antigen contained in the sample from the amount of the antigen bound in the step (b).

(17) The method according to item 16, wherein the chemical formula (II) is represented by the following chemical formula (III).

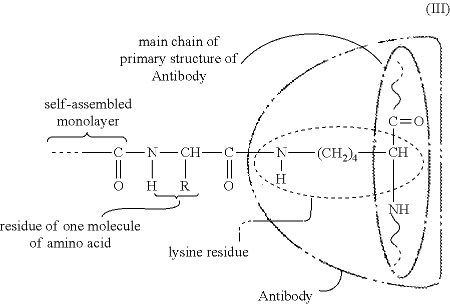

(R represents side chain of one molecule of the amino acid)

(18) The method according to item 16, wherein the one molecule of the amino acid is selected from the group consisting of histidine, cysteine, lysine, phenylalanine, glycine, tryptophan, methionine, serine, asparagine, tyrosine, alanine, glutamic acid, threonine, leucine, valine, and isoleucine.

(19) The method according to item 16, wherein the one molecule of the amino acid is selected from the group consisting of histidine, cysteine, lysine, phenylalanine, glycine, tryptophan, methionine, serine, asparagine, tyrosine, alanine, glutamic acid, and threonine.

(20) The method according to item 16, wherein the one molecule of the amino acid is selected from the group consisting of histidine, cysteine, lysine, phenylalanine, glycine, tryptophan, methionine, serine, asparagine, tyrosine, alanine, and glutamic acid.

(21) The method according to item 16, wherein the one molecule of the amino acid is selected from the group consisting of histidine, cysteine, lysine, phenylalanine, glycine, tryptophan, methionine, serine, asparagine, and tyrosine.

In an exemplary embodiment, the amount of the antibody immobilized per unit area is increased significantly. The benefits and/or advantages may be individually provided by the various embodiments and features of the present disclosure, and need not all be provided in order to obtain one or more of the same.

### BRIEF DESCRIPTION OF DRAWINGS

FIG. 1 shows a schematic view of a method according to the present disclosure.

FIG. 2 is a reproduction of FIG. 7 of Patent Literature 1.

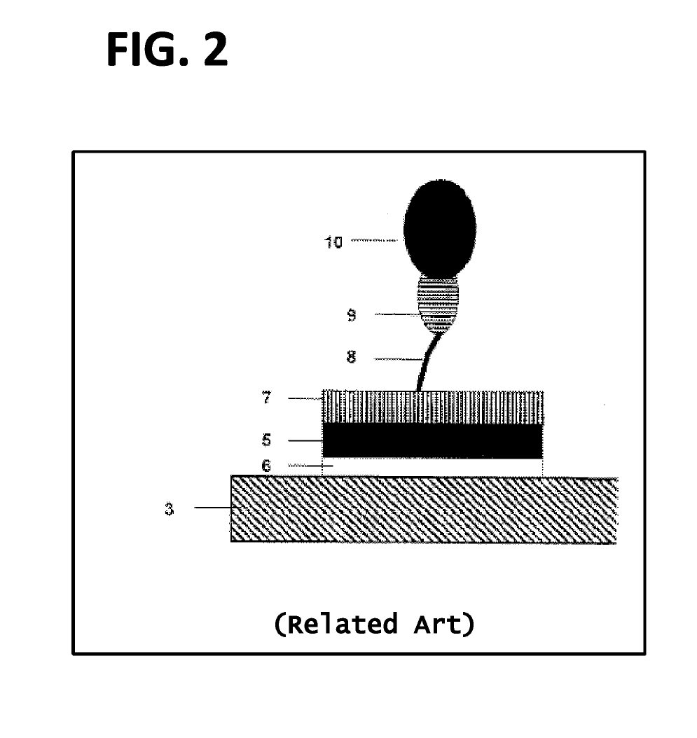

FIG. 3 shows a schematic view of a method according to the related art.

### DESCRIPTION OF EMBODIMENT

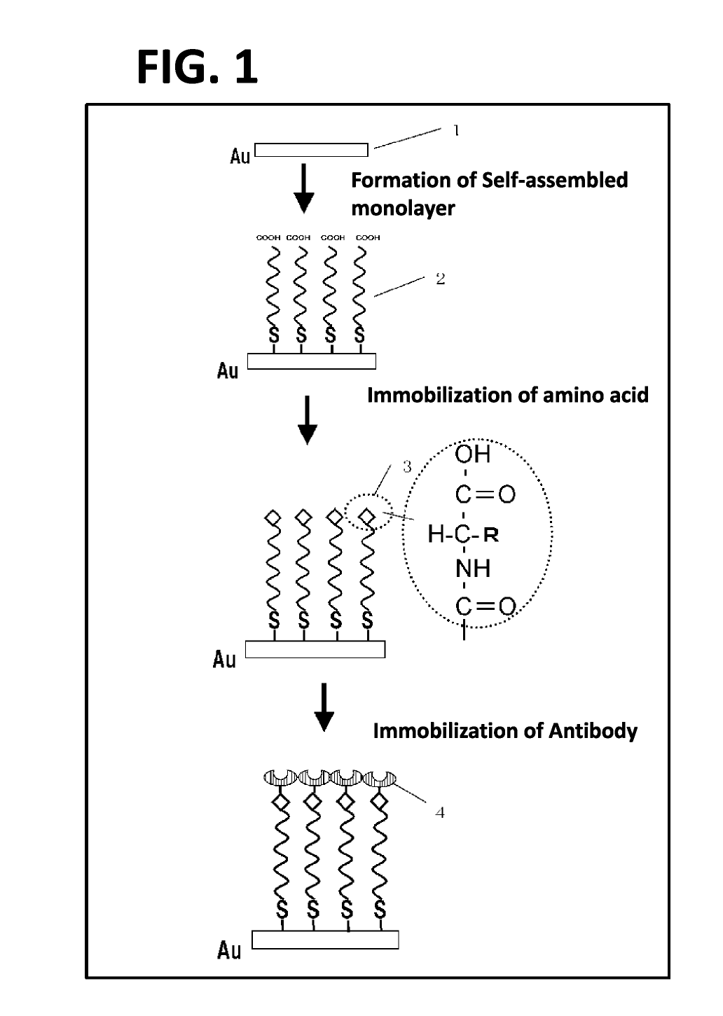

An embodiment of the present disclosure is described below with reference to FIG. 1.

### Embodiment 1

FIG. 1 shows a method according to an embodiment of the present disclosure for immobilizing an antibody on a self-assembled monolayer.

Preferably, a substrate 1 is a gold substrate. An example of the gold substrate is a substrate having a uniform gold layer on its surface. More specifically, the gold substrate may be a substrate having a gold film formed with a sputtering method on the surface of glass, plastic, or SiO₂.

First, the substrate 1 is immersed into a solvent containing alkanethiol molecules. Preferably, the substrate is washed before immersed. Each alkanethiol molecule has a carboxyl group at the end thereof. It is preferable that each alkanethiol molecule has a carbon number falling within the range from six to eighteen. Thus, a self-assembled monolayer 2 is formed on the substrate 1.

The preferred concentration of the alkanethiol molecules is approximately 1 mM to 10 mM. The solvent is not limited as long as it dissolves the alkanethiol molecules. An example of the preferred solvent is ethanol, dimethyl sulfoxide (hereinafter, referred to as “DMSO”), and dioxane. The preferred immersing period is approximately 12 to 48 hours.

Next, an amino acid 3 is supplied to the self-assembled monolayer 2. The carboxyl group (—COOH), which is located at the top end of the self-assembled monolayer 2, reacts with an amino group (—NH₂) of the amino acid 3 to form a peptide bond represented by the following the chemical formula (I).

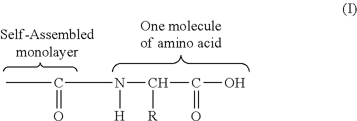

(R represents side chain of one molecule of the amino acid)

In the chemical formula (I), one molecule of the amino acid 3 is bound to the self-assembled monolayer 2.

The amino acid 3 is selected from twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine. Namely, in the chemical formula (I), R represents a side chain of one of amino acid selected from these twenty kinds of amino acids.

When the amino acid 3 is supplied to the self-assembled monolayer 2, two or more kinds of amino acids may be supplied simultaneously. In other words, when a solution containing the amino acid 3 is supplied to the self-assembled monolayer 2, the solution may contain not less than two kinds of the amino acids 3. In light of uniform bind of the antibody to the amino acid 3, which is described later, it is preferred that the solution contains a sole kind of amino acid.

Subsequently, the antibody 4 is supplied. The 5′-terminal amino group of the antibody 4 reacts with the carboxyl group of the amino acid 3. The amino group of the lysine contained in the antibody also reacts with the carboxyl group of the amino acid 3. Thus, two peptide bonds represented by the following chemical formula (II) are formed. In this manner, a sensor is obtained.

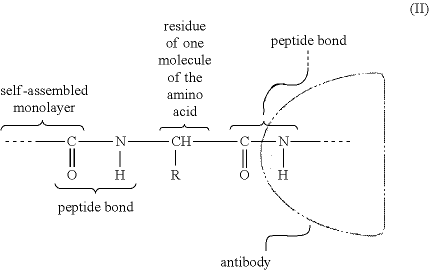

(R represents side chain of one molecule of the amino acid)

One molecule of the antibody 4 has only one 5′-terminal, whereas the one molecule of the antibody 4 has a lot of lysine groups. Therefore, almost all of the chemical formula (II) is represented more specifically by the following chemical formula (III).

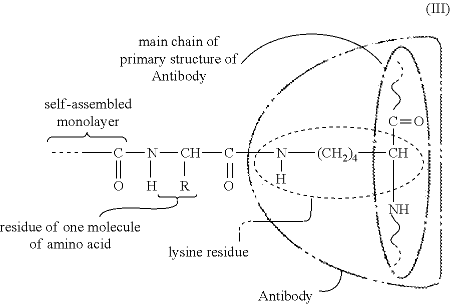

(R represents side chain of one molecule of the amino acid)

The obtained sensor is used for detecting or quantifying the antigen contained in the sample.

More specifically, a sample is supplied to the sensor so as to bind the antigen contained in the sample to the antibody. Needless to say, the antigen is bound specifically to the antibody.

The antigen thus bound is detected or quantified with an ordinal analysis technique such as a Surface Plasmon Resonance (SPR) analysis technique. Another analysis technique such as Quartz Crystal Microbalance (QCM) may be used.

### EXAMPLES

The following examples and a comparative example describe the present technologies in more detail. It is understood that the examples and embodiments described herein are for illustrative purposes only and that various modifications or changes in light thereof will be suggested to persons skilled in the art and are to be included within the spirit and purview of this application and scope of the appended claims.

### Comparative Example

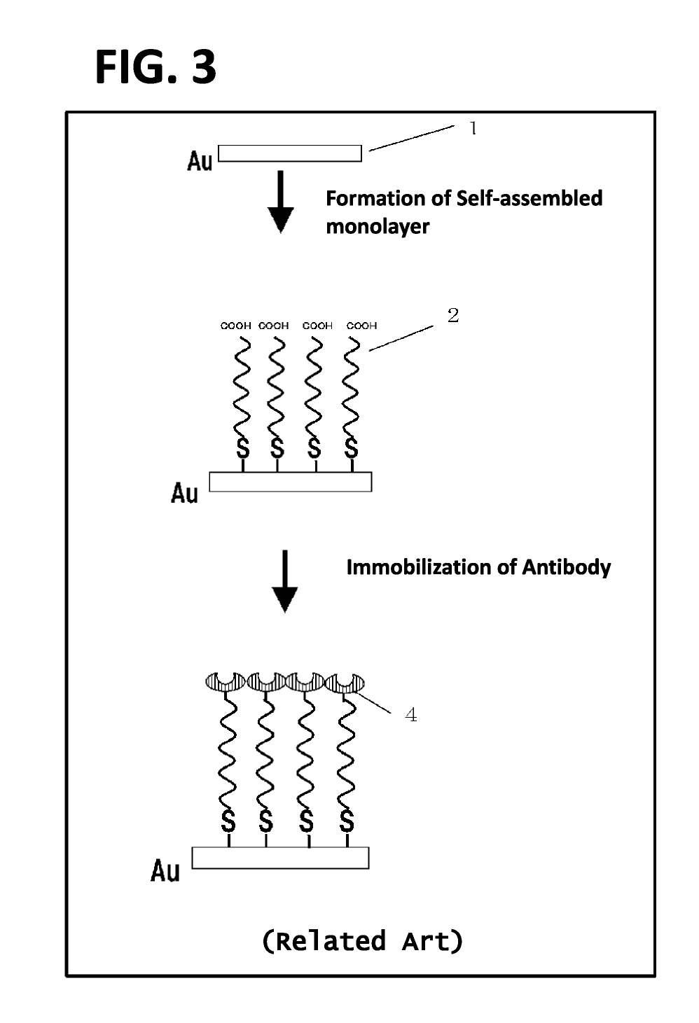

As shown in FIG. 3, the antibody was bound directly with an amide coupling reaction to a carboxyl group located at the top end of self-assembled alkanethiol formed on the gold surface so as to immobilize the antibody. The procedure and the results were described below.

[Preparation of a Sample Solution]

A sample solution of 16-Mercaptohexadecanoic acid with a final concentration of 10 mM was prepared. The solvent thereof was ethanol

[Formation of a Self-Assembled Monolayer]

A gold substrate (available from GE healthcare company, BR-1004-05) having gold vapor-deposited on a glass plate was used as a substrate 1. The substrate 1 was washed for ten minutes with a piranha solution containing concentrated sulfuric acid and 30% hydrogen peroxide water. The volume ratio of the concentrated sulfuric acid to the 30% hydrogen peroxide water contained in the piranha solution was 3:1. Subsequently, the substrate 1 was washed with pure water, and dried.

Next, the gold substrate was immersed in the sample solution for 18 hours to form a self-assembled monolayer on the surface of the gold substrate. Finally, the substrate 1 was washed with pure water and dried.

[Immobilization of the Antibody]

The antibody was bound to the carboxyl group located at the top end of the 16-Mercaptohexadecanoic acid which formed the self-assembled monolayer to immobilize the antibody.

Specifically, the carboxyl group located at the top end of the 16-Mercaptohexadecanoic acid was activated with use of 35 microliters of a mixture of 0.1M NHS (N-Hydroxysuccinimide) and 0.4M EDC (1-ethyl-3-(3-dimethylaminopropyl)carbodiimide hydrochloride). Subsequently, thirty-five microliters of the antibody (2.5 microgram/millimeter) was added at the flow rate of five microliters/minute. Thus, the carboxyl group of the 16-Mercaptohexadecanoic acid was coupled with the amino group of the antibody.

### Example 1

Experiment was conducted similarly to the comparative example except that glycine was supplied as the one molecule of the amino acid between the formation of the self-assembled monolayer and the immobilization of the antibody. The procedure and the results are described below.

[Immobilization of Amino Acid (Glycine)]

Glycine was bound with the carboxyl group located at the top end of the 16-Mercaptohexadecanoic acid which formed the self-assembled monolayer 2 to immobilize the glycine.

More specifically, after the carboxyl group was activated similarly to the comparative example, thirty-five microliters of 0.1M glycine (pH:8.9) was added at the flow rate of 5 microliters/minute. Thus, the carboxyl group of 16-Mercaptohexadecanoic acid was coupled with the amino group of the glycine.

[Immobilization of the Antibody]

Subsequently, the antibody was bound to the carboxyl group of the glycine to immobilize the antibody. More specifically, after the carboxyl group of the glycine was activated similarly to the above, thirty-five microliters of the antibody (concentration: 2.5 micrograms/nil) was added at the flow rate of 5 microliters/minute. Thus, the carboxyl group was coupled with the 5′-terminal amino acid of the antibody or with the amino group of the lysine contained in the antibody.

[Comparison of the Immobilization Amounts]

The immobilization amounts in the example 1 and in the comparative example were measured with use of a Surface Plasmon Resonance (SPR) device, Biacore 3000 (available from GE healthcare company). As used herein, the term “immobilization amount” means the amount of the antibody immobilized per unit area. The ratio of the immobilization amount measured in the example 1 to the immobilization amount measured in the comparative example was approximately 18:1.

### Other Examples

Threonine, methionine, isoleucine, proline, serine, glutamine, asparagine, phenylalanine, tryptophan, cysteine, histidine, alanine, lysine, leucine, glutamic acid, valine, aspartic acid, argnine, and tyrosine were used instead of glycine to measure the respective immobilization amounts similarly to the example 1. These amino acids are twenty kinds of natural amino acid. Table 1 shows the resultant immobilization amounts.

TABLE 1

| | | |
|---|---|---|
| Histidine | 23.86045 | |
| Cysteine | 22.74856 | |
| Lysine | 20.91865 | |
| Phenylalanine | 18.86891 | |
| Glycine | 18.63296 | |
| Tryptophan | 17.46708 | |
| Methionine | 16.50562 | |
| Serine | 16.01948 | |
| Asparagine | 15.96672 | |
| Tyrosine | 15.85254 | |
| Alanine | 15.40134 | |
| Glutamic acid | 14.41335 | |
| Threonine | 13.00732 | |
| Leucine | 8.816629 | |
| Valine | 5.974514 | |
| Isoleucine | 5.701262 | |
| Aspartic acid | 3.676188 | |
| Proline | 3.276342 | |
| Argnine | 2.457678 | |
| Glutamine | 1.171725 | |
| (None) | 1 | ←Comparative Example |

A skilled person in the art would understand the following matters from Table 1. When the twenty kinds of amino acids were used, the immobilization amounts increase, compared to the comparative example. Furthermore, the immobilization amount changes depending on the employed amino acid.

Histidine, cysteine, lysine, phenylalanine, glycine, tryptophan, methionine, serine, asparagine, tyrosine, alanine, glutamic acid, threonine, leucine, valine, and isoleucine are preferred, because each of the measured immobilization amounts are five or more in a case where one amino acid selected from these amino acids is supplied.

Histidine, cysteine, lysine, phenylalanine, glycine, tryptophan, methionine, serine, asparagine, tyrosine, alanine, glutamic acid, and threonine are preferred, because each of the measured immobilization amounts are ten or more in a case where one amino acid selected from these amino acids is supplied.

Histidine, cysteine, lysine, phenylalanine, glycine, tryptophan, methionine, serine, asparagine, tyrosine, alanine, and glutamic acid are preferred, because each of the measured immobilization amounts are not less than the average value of thirteen in a case where one amino acid selected from these amino acids is supplied.

Histidine, cysteine, lysine, phenylalanine, glycine, tryptophan, methionine, serine, asparagine, and tyrosine are preferred, because each of the measured immobilization amounts 15.6 or more, namely greater than 1.2 fold of the average value of 13, in a case where one amino acid selected from these amino acids is supplied.

### INDUSTRIAL APPLICABILITY

The present disclosure significantly increases the amount of the antibody to be immobilized per unit area. This improves the sensitivity or the accuracy of the biosensor. The biosensor may be used for an inspection or a diagnosis which requires the detection or the quantification of the antigen contained in the living sample derived from a patient at a clinical practice.

What we claim is:

1. A method for immobilizing an antibody on a self-assembled monolayer, the method comprising steps of:
a step (a) of preparing a substrate comprising one molecule of an amino acid and the self-assembled monolayer, wherein
the one molecule of the amino acid is bound to the self-assembled monolayer through a peptide bond represented by the following chemical formula (I): 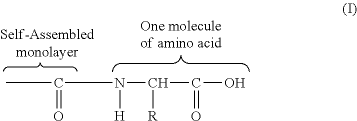
(R represents side chain of one molecule of the amino acid)
the one molecule of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine, and
a step (b) of supplying the antibody to the substrate to form a peptide bond represented by the following chemical formula (II) as a result of reaction between the carboxyl group of the one molecule of the amino acid and the amino group of the antibody: 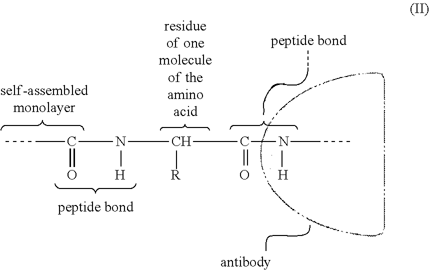
(R represents side chain of one molecule of the amino acid).

2. The method according to claim 1, wherein the step (a) comprises the following steps (a1) and (a2):
a step (a1) of preparing a substrate comprising a self-assembled monolayer on the surface thereof, the self-assembled monolayer having a carboxyl group at one end, and
a step (a2) of supplying the one molecule of the amino acid to form a peptide bond represented by the chemical formula (I) as a result of reaction between the carboxyl group of the one end of the self-assembled monolayer and the amino group of the one molecule of the amino acid.

3. The method according to claim 1, further comprising the following step (ab) between the step (a) and the step (b):
a step (ab) of activating the carboxyl group of the one molecule of the amino acid with a mixture of N-Hydroxysuccinimide and 1-ethyl-3-(3-dimethylaminopropyl)carbodiimide hydrochloride.

4. The method according to claim 2, further comprising the following step (a1a) between the step (a1) and the step (a2):
a step (a1a) of activating the carboxyl group of the self-assembled monolayer with a mixture of N-Hydroxysuccinimide and 1-ethyl-3-(3-dimethylaminopropyl)carbodiimide hydrochloride.

5. The method according to claim 1, wherein the chemical formula (II) is represented by the following chemical formula (III): 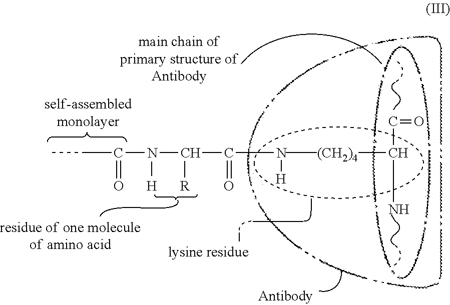
(R represents side chain of one molecule of the amino acid).

6. The method according to claim 1, wherein the one molecule of the amino acid is selected from the group consisting of histidine, cysteine, lysine, phenylalanine, glycine, tryptophan, methionine, serine, asparagine, tyrosine, alanine, glutamic acid, threonine, leucine, valine, and isoleucine.

7. The method according to claim 1, wherein the one molecule of the amino acid is selected from the group consisting of histidine, cysteine, lysine, phenylalanine, glycine, tryptophan, methionine, serine, asparagine, tyrosine, alanine, glutamic acid, and threonine.

8. The method according to claim 1, wherein the one molecule of the amino acid is selected from the group consisting of histidine, cysteine, lysine, phenylalanine, glycine, tryptophan, methionine, serine, asparagine, tyrosine, alanine, and glutamic acid.

9. The method according to claim 1, wherein the one molecule of the amino acid is selected from the group consisting of histidine, cysteine, lysine, phenylalanine, glycine, tryptophan, methionine, serine, asparagine, and tyrosine.

10. A sensor comprising a self-assembled monolayer, one molecule of an amino acid, and an antibody, wherein
the one molecule of the amino acid is interposed between the self-assembled monolayer and the antibody,
the antibody is bound to the self-assembled monolayer through two peptide bonds represented by the following chemical formula (II): 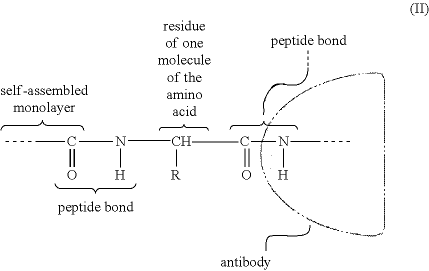
(R represents side chain of one molecule of the amino acid),
the one molecule of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine, and valine.

11. The sensor according to claim 10, wherein the chemical formula (II) is represented by the following chemical formula (III): 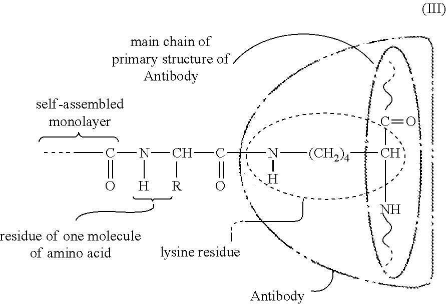
(R represents side chain of one molecule of the amino acid).

12. The sensor according to claim 10, wherein the one molecule of the amino acid is selected from the group consisting of histidine, cysteine, lysine, phenylalanine, glycine, tryptophan, methionine, serine, asparagine, tyrosine, alanine, glutamic acid, threonine, leucine, valine, and isoleucine.

13. The sensor according to claim 10, wherein the one molecule of the amino acid is selected from the group consisting of histidine, cysteine, lysine, phenylalanine, glycine, tryptophan, methionine, serine, asparagine, tyrosine, alanine, glutamic acid, and threonine.

14. The sensor according to claim 10, wherein the one molecule of the amino acid is selected from the group consisting of histidine, cysteine, lysine, phenylalanine, glycine, tryptophan, methionine, serine, asparagine, tyrosine, alanine, and glutamic acid.

15. The sensor according to claim 10, wherein the one molecule of the amino acid is selected from the group consisting of histidine, cysteine, lysine, phenylalanine, glycine, tryptophan, methionine, serine, asparagine, and tyrosine.

16. A method for detecting or quantifying an antigen contained in a sample with use of a sensor, the method comprising steps of:
a step (a) of preparing the sensor comprising a self-assembled monolayer, one molecule of an amino acid, and an antibody, wherein
the one molecule of the amino acid is interposed between the self-assembled monolayer and the antibody,
the antibody is bound to the self-assembled monolayer through two peptide bonds represented by the following chemical formula (II): 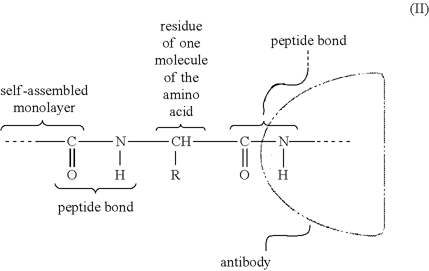
(R represents side chain of one molecule of the amino acid),
the one molecule of the amino acid is selected from the twenty kinds of amino acids consisting of cysteine, lysine, histidine, phenylalanine, tyrosine, glycine, asparagine, methionine, serine, tryptophan, leucine, glutamine, alanine, isoleucine, threonine, proline, glutamic acid, aspartic acid, argnine and valine,
a step (b) of supplying the sample to the sensor to bind the antigen to the antibody, and
a step (c) of detecting the antigen bound in the step (b) or quantify the antigen contained in the sample from the amount of the antigen bound in the step (b).

17. The method according to claim 16, wherein the chemical formula (II) is represented by the following chemical formula (III): 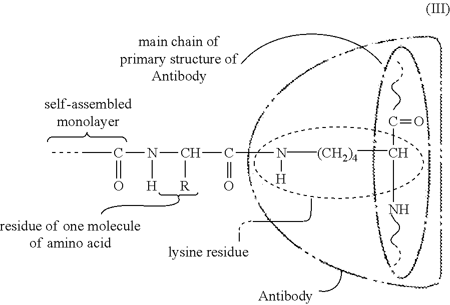
(R represents side chain of one molecule of the amino acid).

18. The method according to claim 16, wherein the one molecule of the amino acid is selected from the group consisting of histidine, cysteine, lysine, phenylalanine, glycine, tryptophan, methionine, serine, asparagine, tyrosine, alanine, glutamic acid, threonine, leucine, valine, and isoleucine.

19. The method according to claim 16, wherein the one molecule of the amino acid is selected from the group consisting of histidine, cysteine, lysine, phenylalanine, glycine, tryptophan, methionine, serine, asparagine, tyrosine, alanine, glutamic acid, and threonine.

20. The method according to claim 16, wherein the one molecule of the amino acid is selected from the group consisting of histidine, cysteine, lysine, phenylalanine, glycine, tryptophan, methionine, serine, asparagine, tyrosine, alanine, and glutamic acid.

21. The method according to claim 16, wherein the one molecule of the amino acid is selected from the group consisting of histidine, cysteine, lysine, phenylalanine, glycine, tryptophan, methionine, serine, asparagine, and tyrosine.
